(19) United States
(12) Patent Application Publication
(10) Pub. No.: US 2010/0105598 A1
(43) Pub. Date: Apr. 29, 2010

(54) Non-Phosphate Dish Detergents

(76) Inventors: Pieter Augustinus, Gouda (NL); Ayrookaran J. Poulose, Belmont, CA (US); Amr R. Toppozada, San Francisco, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/445,313

(22) PCT Filed: Oct. 12, 2007
(86) PCT No.: PCT/US07/21977

§ 371 Date: Oct. 16, 2009

Related U.S. Application Data
(60) Provisional application No. 60/852,042, filed on Oct. 16, 2006.

(51) Int. Cl. C11D 3/386; C12N 9/48

(52) U.S. Cl. 510/226; 435/212

(57) ABSTRACT

The present invention provides non-phosphate containing dishwashing detergent compositions. The present invention also provides methods for the production of and use of such detergents.

### CROSS-REFERENCE TO RELATED APPLICATIONS

This application claims priority to U.S. provisional application 60/852,042, filed Oct. 16, 2006, herein incorporated by reference in its entirety.

### FIELD OF THE INVENTION

The present invention provides non-phosphate containing dishwashing detergent compositions. The present invention also provides methods for the production of and use of such detergents.

### BACKGROUND OF THE INVENTION

Machine dishwashing detergents are formulated as mixtures of ingredients that act to emulsify and remove food soils from dishware, inhibit foam caused by certain food soils, promote the wetting of dishware to minimize or eliminate visually observable spotting, remove stains (e.g., coffee and/or tea), reduce or eliminate tarnishing of flatware, prevent the buildup of soil films on dishware, and/or maximize gentle treatment of the dishware.

Machine dishwashing formulations typically contain approximately five basic ingredients, namely alkalinity carriers, complexing agents, bleaching components, bio-agents (e.g., enzymes), and wetting agents. These formulations also usually contain inorganic phosphate salts as builders to sequester calcium and magnesium ions in water. This sequestration helps minimize filming of dishware. However, because of environmental considerations associated the use of phosphates as builders, various formulations have been developed that do not contain phosphate and/or chlorine. Generally, non-phosphate containing formulations contain salts of low molecular weight inorganic acids (e.g., sodium citrate) as builders. Because citrate is not as effective as phosphate, other additives (e.g., polymers of acrylic acid) are also included in order to minimize the increased spotting and filming that typically occurs with non-phosphate detergent formulations.

Indeed, much effort has been made to replace all or at least some of the phosphates used in dishwashing detergents with chemicals that are more ecologically acceptable. However, very few chemicals have provided promising results. Many chemicals lack the desired cleaning ability, while others lack the building effect of the phosphates, others are less ecologically desirable than phosphates, and some are too expensive to be practical.

Thus, what is needed are dishwashing detergents that do not contain phosphates, but that are as effective as phosphate-containing detergents in soil removal from dishware. In addition, there remains a need for dishwashing compositions that are more environmentally and consumer friendly and are in a form that is easy to use and cost-effective.

### SUMMARY OF THE INVENTION

The present invention provides non-phosphate containing dishwashing detergent compositions. The present invention also provides methods for the production of and use of such detergents. Addition of enzymes or the use of higher active enzymes can over come the cleaning negatives found in formulations that do not contain phosphates. The present invention provides dish detergent compositions that do not contain phosphates. The present invention also provides methods for the production of and use of such detergents. In some particularly preferred embodiments, the present invention provides dish detergent compositions that contain from about 2 to about 3 times the concentration of cleaning enzyme(s) than commonly used detergents.

The present invention also provides non-phosphate containing dishwashing detergents, wherein the detergents comprise at least one protease, wherein said protease is a protease with specific performance greater than twice that of PROPERASE® protease enzyme, and wherein the detergent provides a wash liquor pH between about 7 and about 10.5. In some preferred embodiments, the protease is a subtilisin protease. In some yet further embodiments, the protease comprises at least about 0.02% of said detergent. In some still further preferred embodiments, the detergent further comprises bleaching agents or bleach activators. In some additional preferred embodiments, the detergent further comprises at least one enzyme selected from proteases (including, but not limited to those proteases classified in EC 3.4.21), metalloproteases (including, but not limited to those metalloproteases classified in EC 3.2.24), carbohydrases, oxido-reductases, lipases, pectinases, mannanases, amylases, hemicellulases, esterases, transferases, and perhydrolases.

In some further preferred embodiments, the detergents of the present invention comprise from about 0.13% active protein to about 0.39% active protein (i.e., cleaning enzyme). However, it is not intended that the present invention be limited to any particular percentage of active protein, as any cleaning enzyme concentration that provides the desired cleaning benefit in a detergent that does not contain phosphate finds use in the present invention.

In some particularly preferred embodiments, the cleaning enzyme is PROPERASE® protease, while in some other preferred embodiments, the cleaning enzyme is a wild-type subtilisin or any other suitable subtilisin-type enzyme. Indeed, it not intended that the present invention be limited to the PROPERASE® enzyme, as other enzymes find use in the present invention.

It is also intended that any of the embodiments described herein will find use in any suitable combination. Thus, it is intended that the scope of the present invention encompass all workable combinations of the embodiments described herein.

### DESCRIPTION OF THE INVENTION

The present invention provides non-phosphate containing dishwashing detergent compositions. The present invention also provides methods for the production of and use of such detergents.

Unless otherwise indicated, the practice of the present invention involves conventional techniques commonly used in molecular biology, microbiology, protein purification, protein engineering, protein and DNA sequencing, recombinant DNA fields, and industrial enzyme use and development, all of which are within the skill of the art. All patents, patent applications, articles and publications mentioned herein, both supra and infra, are hereby expressly incorporated herein by reference.

Furthermore, the headings provided herein are not limitations of the various aspects or embodiments of the invention which can be had by reference to the specification as a whole. Accordingly, the terms defined immediately below are more fully defined by reference to the specification as a whole. Nonetheless, in order to facilitate understanding of the invention, definitions for a number of terms are provided below.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Margham, The Harper Collins Dictionary of Biology, Harper Perennial, N.Y. (1991) provide those of skill in the art with a general dictionaries of many of the terms used in the invention. Although any methods and materials similar or equivalent to those described herein find use in the practice of the present invention, preferred methods and materials are described herein. Accordingly, the terms defined immediately below are more fully described by reference to the Specification as a whole. Also, as used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “compatible,” means that the cleaning composition materials do not reduce the enzymatic activity of the protease enzyme(s) provided herein to such an extent that the protease(s) is/are not effective as desired during normal use situations. Specific cleaning composition materials are exemplified in detail hereinafter.

As used herein, “effective amount of enzyme” refers to the quantity of enzyme necessary to achieve the enzymatic activity required in the specific application. Such effective amounts are readily ascertained by one of ordinary skill in the art and are based on many factors, such as the particular enzyme variant used, the cleaning application, the specific composition of the cleaning composition, and whether a liquid or dry (e.g., granular) composition is required, and the like.

As used herein, the phrase “detergent stability” refers to the stability of a detergent composition.

In some embodiments, the stability is assessed during the use of the detergent, while in other embodiments, the term refers to the stability of a detergent composition during storage.

As used herein, the terms “purified” and “isolated” refer to the removal of contaminants from a sample. For example, an enzyme of interest is purified by removal of contaminating proteins and other compounds within a solution or preparation that are not the enzyme of interest. In some embodiments, recombinant enzymes of interest are expressed in bacterial or fungal host cells and these recombinant enzymes of interest are purified by the removal of other host cell constituents; the percent of recombinant enzyme of interest polypeptides is thereby increased in the sample.

As used herein, “protein of interest,” refers to a protein (e.g., an enzyme or “enzyme of interest”) which is being analyzed, identified and/or modified. Naturally-occurring, as well as recombinant (e.g., mutant) proteins find use in the present invention.

As used herein, “protein” refers to any composition comprised of amino acids and recognized as a protein by those of skill in the art. The terms “protein,” “peptide” and polypeptide are used interchangeably herein. Wherein a peptide is a portion of a protein, those skilled in the art understand the use of the term in context.

As used herein, functionally and/or structurally similar proteins are considered to be “related proteins.” In some embodiments, these proteins are derived from a different genus and/or species, including differences between classes of organisms (e.g., a bacterial protein and a fungal protein). In some embodiments, these proteins are derived from a different genus and/or species, including differences between classes of organisms (e.g., a bacterial enzyme and a fungal enzyme). In additional embodiments, related proteins are provided from the same species. Indeed, it is not intended that the present invention be limited to related proteins from any particular source(s). In addition, the term “related proteins” encompasses tertiary structural homologs and primary sequence homologs (e.g., the enzymes of the present invention). In further embodiments, the term encompasses proteins that are immunologically cross-reactive.

As used herein, the terms “detergent composition” and “detergent formulation” are used in reference to mixtures which are intended for use in a wash medium for the cleaning of soiled objects. In preferred embodiments, the term is used in reference to detergents used to clean dishes, cutlery, etc. (e.g., “dish detergents” or “dishwashing detergents”). It is not intended that the present invention be limited to any particular detergent formulation or composition. Indeed, it is intended that in addition to detergents that contain at least one protease of the present invention, the term encompasses detergents that contain surfactants, transferase(s), hydrolytic enzymes, oxido reductases, builders, bleaching agents, bleach activators, bluing agents and fluorescent dyes, caking inhibitors, masking agents, enzyme activators, antioxidants, and solubilizers.

As used herein, “dishwashing composition” refers to all forms of compositions for cleaning dishware, including cutlery, including but not limited to granular and liquid forms. It is not intended that the present invention be limited to any particular type or dishware composition. Indeed, the present invention finds use in cleaning dishware (e.g., dishes, including, but not limited to plates, cups, glasses, bowls, etc.) and cutlery (e.g., utensils, including but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, “non-phosphate containing dishwashing detergents” are detergents that contain no more than 0.5% phosphorus (i.e., phosphorus is a trace element).

As used herein, “wash performance” of mutant protease refers to the contribution of a mutant protease enzyme to dishwashing that provides additional cleaning performance to the detergent without the addition of the mutant protease to the composition. Wash performance is compared under relevant washing conditions.

The term “relevant washing conditions” is used herein to indicate the conditions, particularly washing temperature, time, washing mechanics, sud concentration, type of detergent and water hardness, actually used in households in a dish detergent market segment.

The term “improved wash performance” is used to indicate that a better end result is obtained in stain removal from dishware and/or cutlery under relevant washing conditions, or that less mutant protease, on weight basis, is needed to obtain the same end result relative to the corresponding wild-type enzyme.

The term “retained wash performance” is used to indicate that the wash performance of a mutant protease enzyme, on weight basis, is at least 80% relative to the corresponding wild-type protease under relevant washing conditions.

Wash performance of proteases is conveniently measured by their ability to remove certain representative stains under appropriate test conditions. In these test systems, other relevant factors, such as detergent composition, sud concentration, water hardness, washing mechanics, time, pH, and/or temperature, can be controlled in such a way that conditions typical for household application in a certain market segment are imitated. The laboratory application test system described herein is representative for household application when used on proteolytic enzymes modified through DNA mutagenesis. Thus, the methods provided herein facilitate the testing of large amounts of different enzymes and the selection of those enzymes which are particularly suitable for a specific type of detergent application. In this way “tailor made” enzymes for specific application conditions are easily selected.

As used herein, the terms “protease,” and “proteolytic activity” refer to a protein or peptide exhibiting the ability to hydrolyze peptides or substrates having peptide linkages. Many well known procedures exist for measuring proteolytic activity. For example, in some embodiments, proteolytic activity is ascertained by comparative assays which analyze the respective protease's ability to hydrolyze a commercial substrate. Exemplary substrates useful in the analysis of protease or protelytic activity, include, but are not limited to di-methyl casein, bovine collagen, bovine elastin, and bovine keratin. Colorimetric assays utilizing these substrates are well known in the art (See e.g., WO 99/34011; and U.S. Pat. No. 6,376,450, both of which are incorporated herein by reference). The pNA assay (See e.g., Del Mar et al., Anal. Biochem., 99:316-320 [1979]) also finds use in determining the active enzyme concentration for fractions collected during gradient elution. This assay measures the rate at which p-nitroaniline is released as the enzyme hydrolyzes the soluble synthetic substrate, succinyl-alanine-alanine-proline-phenylalanine-p-nitroanilide (sAAPF-pNA). The rate of production of yellow color from the hydrolysis reaction is measured at 410 nm on a spectrophotometer and is proportional to the active enzyme concentration. In addition, absorbance measurements at 280 nm can be used to determine the total protein concentration. The active enzyme/total-protein ratio gives the enzyme purity.

As used herein, the term “comparative performance” in the context of cleaning activity refers to at least about 60%, at least about 70%, at least about 80% at least about 90%, or at least about 95% of the cleaning activity of a comparative subtilisin protease (e.g., commercially available proteases), including but not limited to OPTIMASE™ protease (Genencor), PURAFECT® protease products (Genencor), SAVINASE® protease (Novozymes), BPN'-variants, and GG36-variants (See e.g., U.S. Pat. No. Re 34,606), RELASE™, DURAZYME™, EVERLASE®, KANNASE™ protease (Novozymes), MAXACAL™, MAXAPEMT™, PROPERASE®, and PURAMAX™ proteases (Genencor; See also, U.S. Pat. No. Re 34,606, U.S. Pat. Nos. 5,700,676; 5,801,038; 5,955,340; 5,972,682; 6,218,165; 6,287,841; 6,312,936; 6,465,235; 6,482,628; 6,586,221; 6,815,193; 7,129,076; EP 130 756; EP 328 229; EP 571 049; and EP 723 590), and B. lentus variant protease products [for example those described in WO 92/21760, WO 95/23221 and/or WO 97/07770 (Henkel). Exemplary subtilisin protease variants include, but are not limited to those having substitutions or deletions at residue positions equivalent to positions 76, 87, 101, 103, 104, 118, 120, 129, 130, 159, 167, 170, 194, 195, 217, 232, 235, 236, 245, 248, and/or 252 of BPN' (e.g., PROPERASE® protease comprises the GG36 protease sequence as known in the art, with the substitutions 87N, 101G, and 104N, using BPN' numbering, as known in the art). In some embodiments, cleaning performance is determined by comparing the proteases of the present invention with those subtilisin proteases in various cleaning assays concerning enzyme sensitive stains as determined by usual spectrophotometric or analytical methodologies after standard wash cycle conditions.

As used herein, the term “specific performance” refers to the cleaning of specific stains per unit of active protein. In some preferred embodiments, the specific performance is determined using stains such as egg yolk, egg/milk, minced meat, tea, milk, porridge, etc. In some particularly preferred embodiments, the protease used in the non-phosphate dishwashing detergent of the present invention has at least about twice the specific performance of the commercially available PROPERASE® protease (Genencor).

As used herein, the term “disinfecting” refers to the removal of contaminants from the surfaces, as well as the inhibition or killing of microbes on the surfaces of items. It is not intended that the present invention be limited to any particular surface, item, or contaminant(s) or microbes to be removed.

Some bacterial serine proteases are referred to as “subtilisins.” Subtilisins comprise the serine proteases of Bacillus subtilis, Bacillus amyloliquefaciens (“subtilisin BPN'”), and Bacillus licheniformis (“subtilisin Carlsberg”) (See e.g., Markland and Smith, in Boyer (ed.), Enzymes, The (Boyer, ed.) vol. 3, pp. 561-608, Academic Press, New York, [1971]). Bacillus strains such as alkalophilic Bacillus strains produce other proteases. Examples of the latter category include such serine proteases as MAXACAL® protease (also referred to herein as “PB92 protease”, isolated from Bacillus nov. spec. PB92), and SAVI-NASE® protease. Additional proteases, include but are not limited to PROPERASE® protease.

In some embodiments, the dishwashing detergents of the present invention contain varying concentrations of enzymes.

### EXPERIMENTAL

The following Examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); HCl (hydrochloric acid); aa (amino acid); by (base pair); kb (kilobase pair); kD (kilodaltons); gm (grams); μg and ug (micrograms); mg (milligrams); ng (nanograms); μl and ul (microliters); ml (milliliters); mm (millimeters); nm (nanometers); μm and um (micrometer); M (molar); mM (millimolar); μM and uM (micromolar); U (units); V (volts); MW (molecular weight); sec (seconds); min(s) (minute/minutes); hr(s) (hour/hours); a.p. or ap (active protein); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); EtOH (ethanol); PBS (phosphate buffered saline [150 mM NaCl, 10 mM sodium phosphate buffer, pH 7.2]); SDS (sodium dodecyl sulfate); Tris (tris(hydroxymethyl)aminomethane); TAED (N,N,N′N′-tetraacetylethylenediamine); w/v (weight to volume); v/v (volume to volume); MS (mass spectroscopy); TIGR (The Institute for Genomic Research, Rockville, Md.); AATCC (American Association of Textile and Coloring Chemists); SR (soil or stain removal); STPP (tri-polyphosphate); MGDA (methylglycinediacetic acid); TNC (tri-sodium citrate); WFK (wfk Testgewebe GmbH, Bruggen-Bracht, Germany); Amersham (Amersham Life Science, Inc. Arlington Heights, Ill.); ICN (ICN Pharmaceuticals, Inc., Costa Mesa, Calif.); Pierce (Pierce Biotechnology, Rockford, Ill.); Amicon (Amicon, Inc., Beverly, Mass.); ATCC (American Type Culture Collection, Manassas, Va.); Amersham (Amersham Biosciences, Inc., Piscataway, N.J.); Becton Dickinson (Becton Dickinson Labware, Lincoln Park, N.J.); BioRad (BioRad, Richmond, Calif.); Clontech (CLONTECH Laboratories, Palo Alto, Calif.); Difco (Difco Laboratories, Detroit, Mich.); GIBCO BRL or Gibco BRL (Life Technologies, Inc., Gaithersburg, Md.); Novagen (Novagen, Inc., Madison, Wis.); Qiagen (Qiagen, Inc., Valencia, Calif.); Invitrogen (Invitrogen Corp., Carlsbad, Calif.); Finnzymes (Finnzymes Oy, Espoo, Finland); Macherey-Nagel (Macherey-Nagel, Easton, Pa.); Merieux (Institut Merieux, Codex, FR); Kelco (CP Kelco, Atlanta, Ga.); Genaissance (Genaissance Pharmaceuticals, Inc., New Haven, Conn.); DNA 2.0 (DNA 2.0, Menlo Park, Calif.); MIDI (MIDI Labs, Newark, Del.) InvivoGen (InvivoGen, San Diego, Calif.); Sigma (Sigma Chemical Co., St. Louis, Mo.); Sorvall (Sorvall Instruments, a subsidiary of DuPont Co., Biotechnology Systems, Wilmington, Del.); Stratagene (Stratagene Cloning Systems, La Jolla, Calif.); Roche (Hoffmann La Roche, Inc., Nutley, N.J.); Agilent (Agilent Technologies, Palo Alto, Calif.); Minolta (Konica Minolta, Ramsey, N.J.); Zeiss (Carl Zeiss, Inc., Thornwood, N.Y.); Henkel (Henkel, GmbH, Diisseldorf, Germany); Cognis (Cognis Corp, USA, Cincinnati, Ohio); Finnzymes (Finnzymes Oy, Espoo, Finland); Reckitt Benckiser, Berks, United Kingdom); BASF (BASF Corp., Florham Park, N.J.); and WFK (Testgewebe GmbH, Brüggen-Bracht, Germany).

In the following Examples, PROPERASE® (Genencor) was the enzyme tested. The “1× dosage” used was 0.13% active protein, while the “3× dosage” was 0.39% active protein. It is contemplated that lower enzyme percentages also find use in the present invention, as long as the specific performance is better than PROPERASE®. For example, in some embodiments, the enzyme is present in a detergent at about 0.02% of the detergent formulation. However, it is not intended that the present invention be limited to any particular percentage of protease. As used herein, the term “specific performance” refers to the cleaning of specific stains per unit of active protein. In some preferred embodiments, the specific performance is determined using stains such as egg yolk, egg/milk, minced meat, tea, milk, porridge, etc.

### Example 1

Comparison of Phosphate-Containing Detergent and Non-Phosphate-Containing Detergent

In this Example, experiments conducted to compare the performance of phosphate-containing and non-phosphate detergent formulations are described.

The detergents used in these experiments are described below. These detergents were obtained from the source without the presence of enzymes, to allow analysis of the enzymes tested in these experiments. As indicated above, the enzyme used in these experiments was PROPERASE® protease (Genencor).

Phosphate-Free Detergent
IEC-60436 WFK Type B (pH = 10.4 in 3 g/l)

| Component | Wt % |
|---|---|
| Sodium citrate dihydrate (N 1560; Jungbunzlauer) | 30.0 |
| Maleic acid/acrylic acid copolymer sodium Salt | 12.0 |
| (SOKALAN ® CP5; BASF)* | |
| Sodium perborate monohydrate | 5.0 |
| TAED (Warwick) | 2.0 |
| Sodium disilicate (PORTIL ® A; Cognis) | 25.0 |
| Linear fatty alcohol ethoxylate (Plurafac LF 403; | 2.0 |
| BASF) | |
| Sodium carbonate anhydrous (Soda leicht; Matthes | add to 100 |
| & Weber) | |

*An alternative is 20% Norasol WL4/Norsohaas (30% active on sodium carbonate).

Phosphate-Containing Detergent:
IEC-60436 WFK Type C (pH = 10.5 in 3 g/l))

| Component | wt % |
|---|---|
| Sodium tripolyphosphate (Thermphos NW; | 23.0 |
| Clariant) | |
| Sodium citrate dihydrate (N 1560; Jungbunzlauer) | 22.3 |
| Maleic acid/Acrylic Acis Copolymer Sodium Salt | 4.0 |
| (SOKALAN ® CP5; BASF)* | |
| Sodium perborate monohydrate | 6.0 |
| TAED (Warwick) | 2.0 |
| Sodium disilicate (non-crystalline): Protil A | 5.0 |
| (Cognis) | |
| Linear Fatty Alcohol Ethoxylate (Plurafac LF 403; | 2.0 |
| BASF) | |
| Sodium Carbonate anhydrous (Soda leicht; Matthes | add to 100 |
| & Weber) | |

*An alternative is 20% Norasol WL4/Norsohaas (30% active on sodium carbonate).

### Egg Yolk Stains on Stainless Steel

The stainless steel sheets (10×15 cm; brushed on one side) used in these experiments were thoroughly washed at 95° C. in a laboratory dishwasher with a high-alkalinity commercial detergent (e.g., ECOLAB® detergent; Henkel) to provide sheets that were clean and grease-free. These sheets were deburred prior to their first use. The sheets were dried for 30 minutes at 80° C. in a thermal cabinet before being soiled with egg yolk. The surfaces to be brushed were not touched prior to soiling. Also, no water stains or fluff on the surfaces were permitted. The cooled sheets were weighed before soiling.

The egg yolks were prepared by separating the yolks of approximately 10-11 eggs (200 g of egg yolk) from the whites. The yolks were stirred with a fork in a glass beaker to homogenize the yolk suspension. The yolks were then strained (approx. 0.5 mm mesh) to remove coarse particles and any egg shell fragments.

A flat brush (2.5″) was used to apply 1.0±0.1 g egg yolk suspension as uniformly as possible over an area of 140 cm² on the brushed sides of each of the stainless steel sheets, leaving an approx. 1 cm wide unsoiled rim (adhesive tape was used if needed). The soiled sheets were dried horizontally (to prevent formation of droplets on the edges of the sheets), at room temperature for 4 hours (max. 24 h).

For denaturation, the sheets were immersed for 30 seconds in boiling, demineralized water (using a holding device if necessary). Then, the sheets were dried again for 30 min at 80° C. After drying and cooling, the sheets were weighed. After weighing, the sheets were left for at least 24 hours (20° C., 40-60% relatively humidity) before submitting them to the wash test. In order to meet the testing requirements, only sheets with 500±100 mg/140 cm² (egg yolk after denaturation), were used in the testing. After the wash tests were conducted, the sheets were dried for 30 min at 80° C., in the thermal cabinet, and weighed again after cooling. The percent cleaning performance was determined by dividing the (mg of egg yolk released by washing×100) by the (mg of denatured egg yolk applied).

### Minced Meat on Porcelain Plates

For these experiments, dessert plates (Arzberg, white, glazed porcelain) conforming to EN 50242, form 1495, No. 0219, diameter 19 cm were used. A total of 225 g lean pork and beef (half and half) was finely chopped and cooled, after removing visible fat. The mixture was twice run through a mincer. Temperatures above 35° C. were avoided. Then, 225 g of the minced meat was mixed with 75 g of egg (white and yolk mixed together). The preparation was then frozen up to three months at −18° C., prior to use. If pork was not available, beef was used, as these are interchangeable.

The minced meat and egg mixture (300 g) was brought up to room temperature and mixed with 80 ml synthetic water. The mixture was then homogenized using a kitchen hand blender for 2 min. Then, a fork was used to spread 3 g of the minced meat/egg/water mixture on each white porcelain plate, leaving an approx. 2 cm wide unsoiled margin around the rim. The amount applied was 11.8±0.5 mg/cm². The plates were dried for 2 hours at 120° C. in a preheated thermal cabinet. As soon as the plates were cooled, they were ready for use. The plates were stacked with paper towels between each of the plates.

After washing, the plates were sprayed with ninhydrin solution (1% ethanol) for better identification of the minced meat residues. To promote the color reaction, the plates were heated for 10 min at 80° C. in the thermal cabinet. Evaluation of the washing performance was done by visually inspecting the color reactions of the minced meat residues with reference to the IKW photographic catalogue (IKW).

### Egg/Milk Stains on Stainless Steel

The stainless steel sheets (10×15 cm; brushed on one side) used in these experiments were thoroughly washed at 95° C. in a laboratory dishwasher with a high-alkalinity commercial detergent to remove grease and clean the sheets. The sheets were polished dry with a cellulose cloth. The surfaces to be brushed were not touched prior to soiling. Also, no water stains or fluff on the surfaces were permitted. Before soiling, the sheets were placed in a thermal cabinet at 80° C., for 30 min. The cooled sheets were weighed before soiling.

The egg yolks and whites of whole raw eggs (3-4 eggs; 160 g/egg) were placed in a bowl and beaten with an egg whisk. Then, 50 ml semi-skimmed UHT (1.5% fat, ultra-high temperature, homogenized) milk were added to the mixture. The milk and egg were mixed without generating froth. A flat brush was used to uniformly distribute 1.0±0.1 g of the egg/milk mixture on the brushed side of the stainless steel sheets, using a balance to check the distribution. A margin of approximately 1.0 cm was left around the short sides of the sheets. The soiled sheets were dried horizontally (to prevent formation of droplets on the edges of the sheets), at room temperature for 4 hours (max. 24 h).

The sheets were then immersed for 30 seconds in boiling, demineralized water (using a holding device if necessary). Then, the sheets were dried again for 30 min at 80° C. After drying and cooling, the sheets were weighed. After weighing, the sheets were left for at least 24 hours (20° C., 40-60% relatively humidity), before submitting them to the wash test. In order to meet the testing requirements, only sheets with 190±10 mg egg yolk were used.

After the wash tests were conducted, the sheets were dried for 30 min at 80° C., in the thermal cabinet, and weighed again after cooling. The percentage cleaning performance was determined by dividing the (mg of egg/milk released by washing×100) by the (mg of egg/milk applied).

### Tea Stains on Tea Cups

To prepare tea stains, tea cups having a wall thickness of 6-8 mm (e.g., Bauscher, Art. No. 6215/18) were used. As the cup wall thickness determines the cooling rate of the tea, it was important to consistently use cups with this wall thickness. The cups were thoroughly washed in either a household dishwasher at 65° C., with detergent IEC A or a laboratory dishwasher at 95° C. and commercial detergent, prior to being stained with tea. To prepare the tea for about 20 cups, 2 liters of hardness-enhanced water (i.e., synthetic water with hardness raised to 3.00 mmol (Ca and Mg to 16.8° d, conforming to draft IEC 734, method B)) were mixed with 0.1 ml of ferric sulfate solution (See, below) and brought to a boil. Then, the boiling water was poured onto 30 g of tea (e.g., Assam; Lipton) in an open container and left to draw for 5 min. The tea was then poured through a strainer (mesh width of 0.5 mm) into another temperature-controlled vessel. Assam tea has been noted as being particularly difficult to remove.

The clean cups were filled with 100 ml tea, such that the temperature of the tea in the cups was 85° C. The initial temperature of the poured tea was about 93° C. Every 5 minutes, 20 ml were removed from the cups with a pipette, until all the cups were empty (5 times). This process was repeated once more with freshly brewed tea. Before washing, the soiled cups were stored for at least 3 days in a room with constant conditions (20° C., 60% relative humidity).

It has been shown that when synthetic water is used whose hardness has been supplemented solely with Ca and Mg ions, the tea cups were not stained darkly enough. For this reason, ferric ions were added to the synthetic water to produce a darker tea stain. This ferric sulfate stock solution was prepared by dissolving 5 g Fe₂(SO₄)₃ and 1 ml HCl (37%) in demineralized water and filling to 1 litre. Then, 0.1 ml of the stock was added to 2 liters of tea.

Evaluation of the washing performance was done by visually inspecting the color reactions of the tea residues with reference to the IKW photographic catalogue (IKW).

### Microwaved Milk

These stains were prepared by microwaving semi-skimmed UHT milk (1.5%, ultra-high temperature pasteurized and homogenized milk). The microwave was set to an output of 450 W. To preheat the microwave, six 150 ml short form glass beakers containing 50 ml water were arranged symmetrically around the edge of the rotating plate in the microwave. The beakers were heated for 10 min. Then, 10 ml milk at room temperature were poured into each of 6 glass beakers and arranged them in the same pattern as the beakers of water on the rotating plate and heated. The baking time was 10 min at 450 W. As the actual output of microwave ovens may deviate from the setting, microwaves were checked every three months. Depending on the degree of deviation, the baking time was adapted either with an independent time switch or, in the case of microwave ovens which could be set to the nearest second in the 10 minute range, by varying the oven's own time setting. The baking time was precisely observed in order to ensure that the results were reproducible. The exact baking time (in seconds) was marked on the microwave together with its period of validity. After baking, the milk soil was post-treated for 2 hours at 80° C. in a thermal cabinet with recirculating air.

Evaluation of the washing performance was done by visually inspecting the color reactions of the milk residues with reference to the 1 KW photographic catalogue (1 KW).

### Porridge (Oat Flakes)

These stains were prepared on 23 cm diameter, white glazed porcelain (e.g. those corresponding to EN standard, tableware from Arzberg or similar). To prepare the oat flakes, the porridge oats (50 g porridge oats; e.g., Peter Kölln, tender Kölln flakes) were stirred into 750 ml cold synthetic water and 250 ml pasteurized 1.5% milk. The mixture was uniformly heated and boiled for 10 min, with constant stirring. A brush was then used to evenly spread 3 g of hot porridge on the inner plate surface being careful to keep the rim of the plate free. Approximately 10.6±0.5 mg/cm² were applied per plate area. The soiled plates were dried for 2 h at 80° C. in a thermal cabinet. After the plates were cooled to room temperature, they were ready for use in the washing tests. After washing, the remains of the porridge were evaluated by visual inspection with reference to the photographic catalogue. To facilitate easier identification of the residual porridge soil, the plates were immersed in an iodine solution prepared in accordance with DIN 44990.

### Washing Equipment and Conditions

The washing tests were performed in an automatic dishwasher (Miele: G690SC), equipped with soiled dishes and stainless steel sheets, as described above. A defined amount of the detergent was used, as indicated in the tables of results below. The temperatures tested were 45° C., 55° C. and 65° C. The water hardness was 9° or 21° GH (German hardness) (374 ppm Ca).

As indicated above, after washing, the plates soiled with minced meat, tea, microwaved milk, and oat flakes were visually assessed using a photo rating scale of from 0 to 10, wherein “0” designated a completely dirty plate and “10” designated a clean plate. These values correspond to the stain or soil removal (SR) capability of the enzyme-containing detergent.

The washed stainless steel plates soiled with egg yolk and/or egg yolk milk were analyzed gravimetrically to determine the amount of residual stain after washing. The PB92 mutant protease and PROPERASE® protease and other mutants were tested at a level of between 0 and 20.57 mg/active protein per wash.

The detergents used in these experiments are described above. These detergents were obtained from the source without the presence of enzymes, to allow analysis of the enzymes tested in these experiments.

The results are shown in the following Tables.

TABLE 1
Comparison of Non-Phosphate and Phosphate-Containing
Detergents and Differing Enzyme Concentrations

| | | Non-Phosphate + | Phosphate + | Non-Phosphate + |
|---|---|---|---|---|
| Stain | Ph | 1x Enzyme | 1x Enzyme | 3x Enzyme |
| Egg Yolk | 10 | 35 ± 0.3 | 48 ± 0.1 | 52 ± 1.8 |
| (% SR) | | | | |
| Egg Yolk | 7 | 32 ± 1.2 | 32 ± 0.4 | 49 ± 2.0 |
| (% SR) | | | | |
| Egg Yolk/Milk | 10 | 53 ± 2.0 | 69 ± 0.3 | 75 ± 1.3 |
| (% SR) | | | | |
| Egg Yolk/Milk | 7 | 46 ± 2.0 | 42 ± 2.9 | 62 ± 0.6 |
| (% SR) | | | | |
| Minced Meat | 10 | 5.5 ± 1.5 | 7.7 ± 1.5 | 8.0 ± 0.5 |
| (SR) | | | | |
| Minced Meat | 7 | 4.0 ± 1.0 | 5.8 ± 0.5 | 8.0 ± 0.5 |
| (SR) | | | | |

TABLE 2
Comparison of Non-Phosphate and Phosphate-Containing
Detergents and Differing Enzyme Concentrations

| | | Non-Phosphate + | Phosphate + | Non-Phosphate + |
|---|---|---|---|---|
| Stain | PH | 1x Enzyme | 1x Enzyme | 3x Enzyme |
| Milk | 10 | 3.6 ± 0.5 | 4.6 ± 0.5 | 4.0 ± 0.5 |
| (SR) | | | | |
| Milk | 7 | 3.0 ± 1.0 | 3.0 ± 1.0 | 3.0 ± 1.0 |
| (SR) | | | | |
| Oat flakes | 10 | 3.8 ± 0.6 | 5.0 ± 0.8 | 4.0 ± 0.8 |
| (SR) | | | | |
| Oat flakes | 7 | 2.0 ± 0.2 | 5.5 ± 0.5 | 4.0 ± 0.8 |
| (SR) | | | | |
| Tea | 10 | 4.0 ± 0.0 | 5.0 ± 1.0 | 4.5 ± 1.0 |
| (SR) | | | | |
| Tea | 7 | 3.5 ± 0.0 | 2.0 ± 0.5 | 2.5 ± 1.0 |
| (SR) | | | | |

TABLE 3
Comparison of Non-Phosphate and Phosphate-Containing
Detergents Without Enzymes

| Stain | PH | Non-Phosphate | Phosphate |
|---|---|---|---|
| Milk | 10 | 2.0 ± 0.5 | 3.0 ± 0.0 |
| (SR) | | | |
| Milk | 7 | 1.0 ± 1.0 | 2.5 ± 0.0 |
| (SR) | | | |
| Oat flakes | 10 | 4.0 ± 0.5 | 4.5 ± 0.5 |
| (SR) | | | |
| Oat flakes | 7 | 3.5 ± 0.5 | 3.5 ± 1.0 |
| (SR) | | | |
| Tea | 10 | 4.0 ± 0.0 | 6.0 ± 0.5 |
| (SR) | | | |
| Tea | 7 | 3.5 ± 0.5 | 2.0 ± 0.0 |
| (SR) | | | |
| Egg yolk | 10 | 9.6 ± 0.4 | 8.9 ± 0.1 |
| (% SR) | | | |
| Egg yolk | 7 | 12.3 ± 0.8 | 11.0 ± 0.6 |
| (% SR) | | | |
| Egg yolk milk | 10 | 13.3 ± 0.5 | 12.7 ± 1.0 |
| (% SR) | | | |
| Egg yolk milk | 7 | 15.4 ± 1.9 | 12.3 ± 1.5 |
| (% SR) | | | |
| Minced meat | 10 | 1.5 ± 0.0 | 1.0 ± 0.0 |
| (SR) | | | |
| Minced Meat | 7 | 1.0 ± 0.0 | 2.0 ± 0.0 |
| (SR) | | | |

As indicated by the results in the above Tables, non-phosphate containing detergents performed more poorly than detergents that contained phosphate, particularly at pH 10. These results pertain to enzyme-sensitive soiling, as well as bleach-sensitive soiling. The results also indicate that by adding additional enzymes (i.e., 3×0.13% active protein) to non-phosphate containing detergents, the performance is improved to levels similar to those of phosphate-containing detergents.

These results indicate that the performance of non-phosphate-containing detergents can be improved by the addition of 2-3× enzyme.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

Having described the preferred embodiments of the present invention, it will appear to those ordinarily skilled in the art that various modifications may be made to the disclosed embodiments, and that such modifications are intended to be within the scope of the present invention.

Those of skill in the art readily appreciate that the present invention is well adapted to carry out the objects and obtain the ends and advantages mentioned, as well as those inherent therein. The compositions and methods described herein are representative of preferred embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A non-phosphate containing dishwashing detergent, wherein said detergent comprises at least one protease, wherein said protease is a protease with specific performance greater than twice that of PROPERASE® protease enzyme, and wherein said detergent provides a wash liquor pH between about 7 and about 10.5.

2. A non-phosphate containing dishwashing detergent of claim 1, wherein said protease is a subtilisin protease.

3. The non-phosphate containing dishwashing detergent of claim 1, wherein said protease comprises at least about 0.02% of said detergent.

4. The non-phosphate containing dishwashing detergent of claim 1, wherein said detergent further comprises bleaching agents or bleach activators.

5. The non-phosphate containing dishwashing detergent of claim 1, wherein said detergent further comprises at least one enzyme selected from proteases, metalloproteases, carbohydrases, oxido-reductases, lipases, pectinases, mannanases, amylases, hemicellulases, esterases, transferases, and perhydrolases.
